(19) United States
(12) Patent Application Publication
(10) Pub. No.: US 2017/0037446 A1
(43) Pub. Date: Feb. 9, 2017

(54) METHOD FOR THE MICROBIOLOGICAL DETERMINATION OF TRACES OF ANTIBIOTICS IN LOW VOLUME BIOLOGICAL SAMPLES

(71) Applicant: UNIVERSIDAD DE SANTIAGO DE CHILE, Santiago (CL)

(72) Inventors: Loreto SANHUEZA CHAVEZ, Santiago (CL); Marcela Andrea WILKENS ANWANDTER, Santiago (CL); Claudio Aurelio LAURIDO FUENZALIDA, Santiago (CL)

(73) Assignee: UNIVERSIDAD DE SANTIAGO DE CHILE, Santiago (CL)

(21) Appl. No.: 15/106,616

(22) PCT Filed: Dec. 11, 2014
(86) PCT No.: PCT/CL2014/000072

§ 371 Date: Jun. 20, 2016

(30) Foreign Application Priority Data
Dec. 18, 2013 (CL) 3628-2013

(51) Int. Cl. C12Q 1/18

(52) U.S. Cl. CPC C12Q 1/18; G01N 2333/31

(57) ABSTRACT

Method for the microbiological determination of traces of antibiotics in low volume biological samples, which comprises determining the minimum inhibitory concentration (MIC) of an antibiotic selected from among minocycline, ciprofloxacin, kanamycin, tetracycline and oxacillin against Staphylococcus aureus. The bacteria are grown in Mueller-Hinton broth (MHB) overnight, said cultures subsequently being diluted to 0.5 McFarland standard (1.5×108 cells/mL). Said antibiotics are subsequently added to 96-well plates in a final volume of 200 μL and incubated at 37° C. for 18 hours. Absorbency is determined and the results are expressed as the percentage of inhibition relative to the control containing the biological sample, in turn selected from urine, blood, synovial fluid, aqueous humour of the eye or cerebro spinal fluid (CSF), and the bacterial culture. The curve representing the percentage of inhibition of bacterial growth versus concentration of antibiotic is obtained, and is adjusted by the “Four Parameter Logistic Curve (4PL)”, yielding the concentration values of the antibiotics in the biological sample. The method determines amounts of antibiotic in the biological sample even at ng/mL level.

### FIELD OF THE INVENTION

The present invention relates to a microbiological method for determining traces of antibiotics in low volume biological samples.

### BACKGROUND OF THE INVENTION

Numerous studies have confirmed the role of spinal glia in the development and persistence of chronic pain. Consistent with this, it is possible to propose that pharmacological suppression of glial function in the spinal cord may depress spinal mechanisms of pain. Different drugs have shown that, besides acting as antibiotics (minocycline, amoxicillin, neomycin, ceftriaxone), they exhibit anti-inflammatory activity. This makes drugs useful in studies of chronic pain. Usually, these antibiotics are administered via intrathecal to rats having chronic pain conditions, to a compartment having a very low volume (about 80-100 μL) in these animals. Determining these antibiotics by classical methods such as high performance liquid chromatography (HPLC) is a great challenge, as in this case the sample should be cleared of elements present in the cerebrospinal fluid, such as proteins, peptides or other components preventing their use in HPLC chromatography. Moreover, kinetic studies of antibiotics could not be accomplished, because only one sample for HPLC consumes all the cerebrospinal fluid of the rat.

In addition, this methodology can be used for determining antibiotics in other biological fluids such as urine, blood, synovial fluid, eye aqueous humor, etc.

It is also likely that, using HPLC coupled to mass spectrometry, traces of antibiotics in biological samples or others could be detected, but the volume required for detection is high (in the range of mL). Microbiological method is a simple, sensitive and cost-saving alternative compared with determinations made by the HPLC method.

Specifically, in prior art, an assay to determine blood levels of antibiotic (kanamycin and gentamycin) measured in 40 minutes to 1.5 hours using samples of 0.1-0.6 ml of serum, has been disclosed. The principle is based on the measurement of pH drop in a culture containing a heavy inoculum of test organisms. Bacterial growth is inhibited by antibiotics, resulting in the inhibition of fermentation. The degree of inhibition is reflected in the pH change in the culture medium, which is related to the concentration of added antibiotics. See “Rapid microbiological assay of antibiotics in blood and other body fluids” by S. Faine and D. C. Knight in The Lancet, Aug. 17, 1968, pages 375-378

An assay for screening levels of antibiotics in cow's milk, by using Delvotest SP-NT and Copan Milk Test is also known. These inhibitory assays consist of agar ampoules or microplates containing spores of Bacillus stearothermophilus and nutrients. With the addition of the milk under study, the spores germinate and produce carbonic acid. This acid causes the bromocresol purple indicator in the ampoule to change color from purple to yellow. Thus, a yellow ampoule after incubation indicates a negative sample. The presence of antibiotics inhibits bacterial growth and the color of the agar in the ampoule remains purple, indicating a positive result. The Delvotest SP-NT and the Copan Milk Test were developed from S P Delvotest and Delvotest Milk Control Stations (MCS). See Validation and comparison of the Copan. Milk Test and Delvotest SP-NT for the detection of antimicrobials in milk. Marie-Hélène Le Breton, Marie-Claude Savoy-Perroud Jean-Marc Diserens. Analytica Chimica Acta 586 (1-2): 280-283, (2007).

Another known assay is based on the concentration-dependent variation of the inhibitory effect of antibiotics on reference bacteria, B. subtilis ATCC 6633, S. aureus ATCC 6538p and S. epidermidis ATCC 12228, in a seeded agar. Results were expressed as average inhibition zone (in mm) versus antibiotic concentration. See Application of microbiological assay to determine pharmaceutical equivalence of generic intravenous antibiotics. Andrés F. Zuluaga, María Agudelo, Carlos Rodríguez and Omar Vesga. BMC Clinical Pharmacology 2009, 9: 1 doi: 10, 1186/1472-6904-9-1.

Moreover, CN1858232 discloses a technology for detecting antibiotic residues in agricultural products, particularly a method of detecting microbiological sulfa drugs residues in edible animal tissue developed in kit form. The method comprises preparing a culture medium I and a culture medium II; forming a culture medium with culture medium. I and culture medium II, it uses Bacillus megaterium as work microorganism, neomycin paper sheets for quality control and synergistic trimethoprim solution. The technology provides simple detection, high sensitivity, and long shelf life of the kit.

EP0418113 relates to a microbiological test kit and method for detecting antibacterial compounds in a specimen, particularly useful for food or dairy industry. Said method comprising the step of: a) admixing said species with a viable bacterium exhibiting enhanced sensitivity to said antibacterial compound to form a mixture; b) incubating said mixture under optimal growth conditions; c) diluting said mixture with an aqueous solution of signal generating reagents, specially selected to detect at least one metabolic marker existing in said bacterium; d) incubating said mixture for sufficient time for said time signal to be detected; d) measuring or detecting said signal in said mixture; e) comparing said signal to a control specimen free of said antibacterial compound, which was simultaneously subjected, to steps (a) to (d).

Therefore, it would be advantageous to provide a highly sensitive microbiological method for determining antibiotics, based on low volume biological samples (10-20 μL), without any previous preparation.

### BRIEF DESCRIPTION OF THE FIGURES

FIG. 1 shows the distribution curves for S. aureus growth inhibition (%) depending on the concentration of five antibiotics, assayed three times each. Points are mean±standard deviation (SD).

FIG. 2 shows the distribution curves of E. coli growth inhibition (%) depending on the concentration of five antibiotics tested. It may be seen that E. coli was less sensitive to all antibiotics tested. Therefore, the remaining experiments were conducted using S. aureus (each antibiotic being assayed three times; the points are mean±SD).

FIG. 3 shows the curve resulting from the application of 4PL curve equation to minocycline antibiotic obtained from FIG. 1. From it, FIG. 4 curve was drawn.

FIG. 4 shows the kinetics of minocycline injected into the cisterna magna. It can be seen that the present method. allows quantification of minocycline concentrations in the range of 10-100 ng/ml.

### DETAILED DESCRIPTION OF THE INVENTION

### Example

The minimum inhibitory concentration (MIC) of minocycline, ciprofloxacin, kanamycin, tetracycline and oxacillin antibiotics, was determined by microdilution method in 96-well plates according to the Institute for Clinical Laboratory Standards recommended protocol, versus ATCC Staphylococcus aureus ATCC 6538 and Escherichia coli ATCC 1129. Briefly, bacteria were grown overnight in Mueller-Hinton broth (MHB) and diluted to 0.5 McFarland. (1.5×10⁸ cells/mL) . Different antibiotics were added to bacterial cultures in 96-well plates to a final volume of 200 μL. The plates were incubated at 37° C. for 18 h and absorbance was determined in an ELISA reader (Thermo Labsystems Multiskan, FC model). The results were expressed as percent inhibition relative to the control containing the cerebrospinal fluid and bacterial culture. Of the two strains of microorganisms tested, Staphylococcus was chosen as the most sensitive to minocycline. The results show that it is possible, for example, to determine minocycline in cerebrospinal fluid (CSF) up to ng/mL level.

A curve representing the percentage of bacterial growth inhibition versus antibiotic concentration applied to each well was also obtained.. Subsequently, using a curve fit analysis called “Four Parameter Curve Logistic (4PL)”, the values of antibiotic concentrations in the biological sample were obtained.

### Example

Cerebrospinal fluid (CSF) collection: It was collected by transcutaneous cisternal magna puncture. Minocycline injections were performed by the same route.

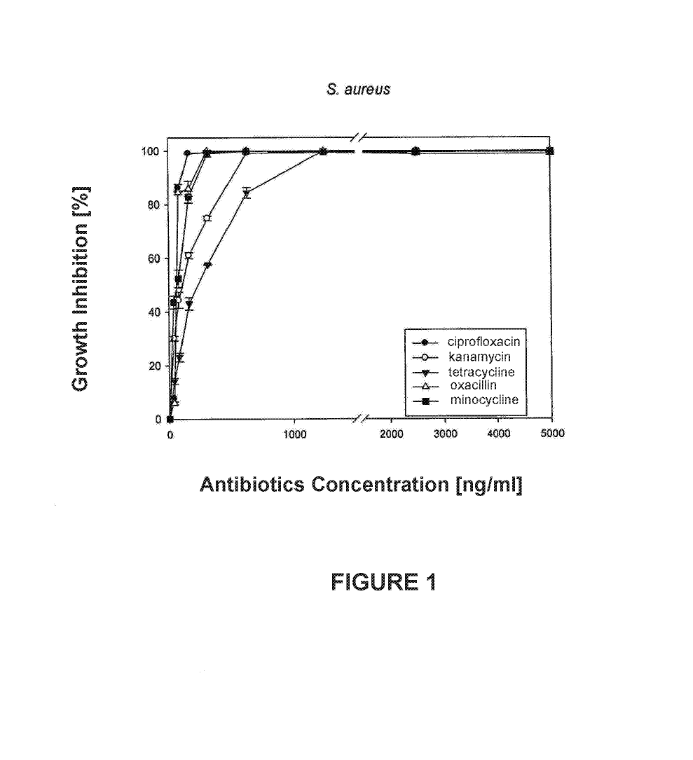

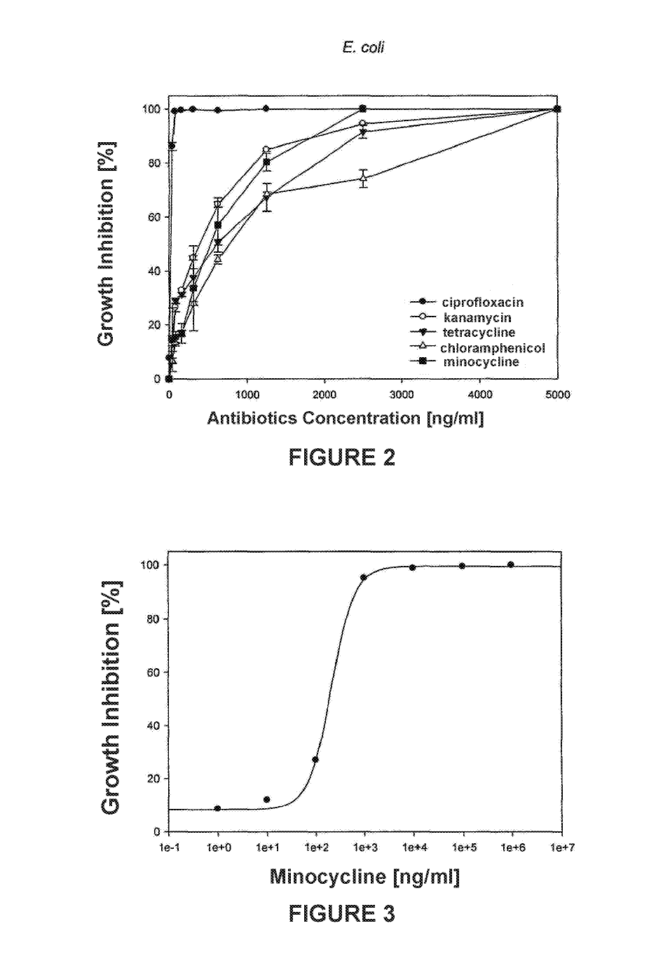

Curve fitting: Four Parameter Logistic Curve (4PL) analysis was applied to minocycline antibiotic obtained from FIG. 1:

y=(max−min)/1+(x/EC₅₀₎ ^{Λ Hillslope}+min, (1)

solving for x:

x=EC₅₀((−max+y)/(min−y)^{Λ(1/Hillslope)} (2)

wherein:

min=Bottom of the curve

max=Upper curve

EC₅₀=The value of x for the curve is halfway between the minimum and maximum parameters. It is called the half-maximum effective concentration.

Hillslope=characterizes the slope of the curve at its midpoint.

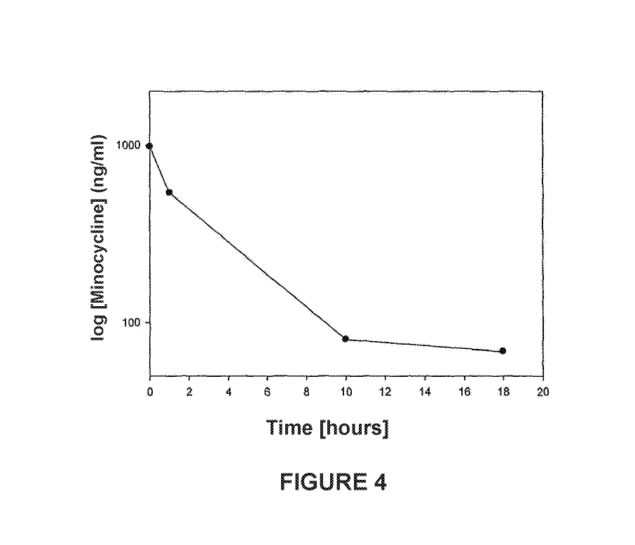

In accordance to the abovementioned, FIG. 4 was generated.

Minocycline (MC), an antibiotic belonging to the family of tetracyclines, was used to carry out pharmacokinetic studies of CSF in Sprague-Dawley rats. Briefly, normal rats were injected via intra CSF an MC solution of 1 mg/mL, for subsequent sampling of CSF at time zero, 1, 10 and 18 hours. It. may be observed that MC is detected at all times. At 18 hours it is still possible to detect a concentration of 69 ng/mL (FIG. 4).

1. A microbiological method for determining traces of antibiotics in low volume biological samples, which comprises determining the minimum inhibitory concentration (MIC) of an antibiotic selected from the group consisting of minocycline, ciprofloxacin, kanamycin, tetracycline and oxacillin versus Staphylococcus aureus, by growing said bacteria separately in Mueller-Hinton broth (MHB), to subsequently dilute said cultures to 0.5 McFarland standard (1.5×10⁸ cells/mL) and adding afterwards said antibiotics and incubating at 37° C. for 18 hours and then determining absorbency and expressing the results as the percentage of inhibition relative to the control containing the biological sample, selected in turn from urine, blood, synovial fluid, eye aqueous humour or cerebrospinal fluid (CSF) , and the bacterial culture; obtaining the curve representing the percentage of bacterial growth inhibition versus antibiotic concentration, and fitting by the “Four Parameter Logistic Curve (4PL)” curve fitting analysis, to yield the antibiotic concentration values in the biological sample.

2. The method of claim 1, wherein said antibiotic is minocycline.

3. The method of claim 1 wherein the biological sample is cerebrospinal fluid.

4. The method of claim 3 wherein the biological sample of cerebrospinal fluid is obtained by transcutaneous cisternal magna puncture of a rat.
